(19) United States
(12) Patent Application Publication
(10) Pub. No.: US 2010/0040706 A1
(43) Pub. Date: Feb. 18, 2010

(54) METHOD OF ANTI-AGEING COSMETIC CARE BY STIMULATION OF SURVIVIN EXPRESSION

(75) Inventors: Marc Dumas, Saint Jean Le Blanc (FR); Frederic Bonte, Orleans (FR); Isabelle Renimel, Trainou (FR)

Correspondence Address: WOODCOCK WASHBURN LLP, CIRA CENTRE, 12TH FLOOR, 2929 ARCH STREET, PHILADELPHIA, PA, 19104-2891, US

(73) Assignee: LVMH RECHERCHE, Saint Jean De Braye (FR)

(21) Appl. No.: 12/477,409

(22) Filed: Jun. 3, 2009

(30) Foreign Application Priority Data
Jun. 6, 2008 (FR) 0853794

(51) Int. Cl. A61K 36/00; A61K 38/00; A61K 31/35

(52) U.S. Cl. 424/725; 514/12; 514/455

(57) ABSTRACT

The invention relates to a cosmetic care method comprising the delivery of an effective amount of at least one cosmetically acceptable agent that activates or stimulates survivin expression in the stem cells of the basal layer of the epidermis. The invention makes it possible in particular to prevent or delay the appearance of the signs of skin aging or to treat them.

### CROSS-REFERENCE TO RELATED APPLICATIONS

This application claims the benefit of French Application No. 0853794, filed Jun. 6, 2008, the entirety of which is incorporated herein.

### TECHNICAL FIELD

The present invention relates to a method of anti-aging cosmetic care by stimulation of survivin expression.

More particularly, the subject of the invention is molecules or extracts, in particular of plant origin, that stimulate survivin expression in the skin, the use thereof as active agents in cosmetic or dermatological compositions, and the cosmetic care or dermatological treatment methods using said compositions.

### BACKGROUND

Apoptosis is an active biological process of elimination, by fragmentation, of certain cells of the organism.

It constitutes a programmed elimination of cells at the biological tissue level, under genetic control. The elimination may be natural (surplus cells in a tissue) or induced by various forms of stress.

The biological cascade of apoptosis is known and uses a number of effectors such as caspases, in particular the effector caspases 3 or 7, which will implement the apoptosis programme, and the initiating caspases 8 and/or 9, which will trigger it.

A certain number of apoptosis inhibitors are also known (Deveraux et al., Genes Dev. 13 (1999), pp. 239-252), among which is survivin. These inhibitors therefore regulate cell survival, thus participating in cell homeostasis in biological tissues.

Survivin, the only member of the IAP (Inhibitor of Apoptosis Protein) family, is a bifunctional protein capable both of balancing the apoptosis of cells and of regulating the cell cycle thereof.

Survivin inhibits in particular the activation of certain caspases, in particular caspases 3, 7 and 9.

This protein is expressed in strongly growing embryonic tissues, but is not expressed in adult differentiated tissues, except in tissues that have a physiological cell renewal and/or are involved in a repair process. Thus, at the cutaneous level, it is most particularly expressed in the keratinocytes of the basal layer of the epidermis, which provide formation and renewal of the latter.

It is in this basal layer that the epidermal stem keratinocytes are found, these being cells with a high potential for regeneration of this tissue, which have been demonstrated to be the most effective in forming a complete epidermis (J L Xie et al., J Plast. Reconstr. Aesthet. Surg. 2007; 60(9): 983-90).

Now, it has been shown that survivin is mainly expressed in the stem cells of the epidermis (Marconi A, Dallaglio K, Lotti R, Vaschieri C, Truzzi F, Fantini F, Pincelli C, Stem cells 2007: 25: 149-155).

Conversely, overexpression of survivin shows a significant decrease in the number of apoptotic cells in the epidermis after exposure to ultraviolet radiation (Grossman et al., 2001 J Clin Invest 108: 991-999).

It has also been demonstrated that the inactivation of beta-1 integrins completely abolishes the cellular expression of survivin (Marconi A et al., Stem cells 2007: 25: 149-155) and leads the cells to apoptosis.

Beta-1 integrins are adhesion proteins through which the keratinocytes of the epidermal basal layer adhere to the proteins of the dermal-epidermal junction.

Beta-1 integrins are expressed more strongly by the stem cells of the epidermis (P. Jones, Cell 1993, 73: 713-724, Kaur J Invest Dermatol 2006, 126, 1450-1458), which corroborates the observation of a stronger expression of survivin in these cells.

Now, during aging, a drop in the expression of beta-1 integrins in the keratinocytes (B Le Varlet et al. J Investig Dermatol Symp Proc. 1998, 3: 172-1 79) and in the wrinkled skin areas exposed to light (S Bosset et al. British J Dermatol 2003, 148: 7770-778) is observed.

Thus, the proteins which ensure maintenance of survivin in the basal cells of the epidermis decrease with age, and, in parallel, an increase in the sensitivity of these cells to apoptosis and a decrease in cycling cells are observed (Zuliani et al., J. Invest. Dermatol. 2004, 123:2, A50, 302), these observations converging to indicate a probable survivin deficiency in aging skin.

In addition to its apoptosis-regulating role, survivin has been identified as a constituent of the “chromosomal passenger complex” which coordinates the chromosomes with cytoskeleton during mitosis (Vader et al., EMBO reports, 2006, 7, 1, 85-92); it therefore plays an essential role in normal cell division, this division being impaired during aging with, as a consequence, less renewal of the epidermis, thinning thereof, and the development of wrinkles.

Survivin is therefore a regulator of the survival and of the resistance of keratinocytes; it acts by modulating the sensitivity of apoptosis of the keratinocytes located in the basal layer of the epidermis, including the stem cells. It also regulates their capacity for renewal and for regeneration of the epidermis.

It thus makes it possible to spare the cell stock of the epidermis and to maintain efficient epidermal cell renewal.

Document WO 2006/069192 (GILLETTE Co) discloses the use, in cosmetics, of survivin-inhibiting agents for a hair and body-hair growth reduction effect.

To date, no compounds that act as survivin-expression stimulators have been described for uses in dermatology or cosmetics.

### SUMMARY

The present invention is directed to methods for caring for a body zone in need thereof, comprising delivering, to at least a part of the body zone, an effective amount of at least one cosmetically acceptable agent that activates or stimulates survivin expression in said body zone.

### DETAILED DESCRIPTION OF ILLUSTRATIVE EMBODIMENTS

The inventors of the present invention had demonstrated that isolated molecules or extracts, more particularly extracts obtained from a material of plant origin, stimulate survivin expression in normal human keratinocyte cultures.

These active agents thus play a protective role with respect to the regenerative cells of the human epidermis and most particularly the stem cells of the basal layer of the epidermis.

These molecules or extracts can be used as an active agent in cosmetic or dermatological compositions aimed in particular at preventing or delaying the appearance of signs of skin aging or reducing the effects thereof, or else at promoting cell or tissue longevity; at promoting the reconstruction of a damaged epidermis and also the healing of cutaneous wounds in normal skin and ulcerative wounds that heal poorly; at preventing or slowing down hair loss, at promoting hair regrowth or hair reinforcement; as an adjuvant for prolonging cell cultures in vitro for the purposes of producing cultured epidermis (reconstructed epidermis) for therapeutic purposes, for example in grafts or else in maintaining purified populations of stem cells of the epidermis or of hair follicles in vitro for therapeutic or research purposes.

The principal objective of the invention is to provide molecules or extracts, in particular of plant origin, that stimulate survivin expression in the skin, the use thereof as active agents in cosmetic or dermatological compositions, and the cosmetic care or dermatological treatment methods using said compositions.

The principal objective of the invention is also to provide a method of anti-aging cosmetic care by stimulation of survivin expression in the skin.

The principal objective of the invention is to propose the use of a cosmetically or dermatologically acceptable molecule or molecules or plant extract obtained from plants, as a cosmetic or dermatological agent.

The objective of the invention is also the use of said molecule or of said extract as a cosmetic or dermatological agent, or as an active agent in cosmetic or dermatological compositions, and the cosmetic care or dermatological methods using said compositions:

a) for preventing or delaying the appearance of the signs of skin aging or slowing down the effects thereof, and/or

b) for reconstructing the epidermis or the stratum corneum thereof, when it is damaged, in particular by ultraviolet radiation, and/or

c) for restoring the functioning of the hair cycle, in order to prevent or slow down hair loss, to accelerate or promote hair regrowth, in particular in the case of alopecia, or to reinforce brittle hair, and/or

d) for promoting growth of the nail and/or reinforcing the strength thereof.

The principal objective of the invention is also to provide a cosmetic care method, using a cosmetically acceptable plant extract, in particular for carrying out the types of cosmetic or dermatological care indicated above.

Finally, the principal objective of the invention is to provide a method for the in vitro culture of stem cells and/or of cells with a high clonogenic potential for the purposes of fundamental studies or of production of cultured epidermis, such as reconstructed epidermis, for therapeutic purposes, for example, as in the case of a graft, following burns or ulcerative wounds which heal poorly, comprising the use of a plant extract that is acceptable with said cell culture, obtained from plants.

A first subject of the invention is a cosmetic care method for the care of a body zone in need thereof, comprising the delivery, to at least a part of the body zone in need thereof, of an effective amount of at least one cosmetically acceptable agent that activates or stimulates survivin expression in said body zone.

According to a first embodiment of the invention, said cosmetic care method comprises the application, to at least a part of the skin of the face or of the body exhibiting or liable to exhibit signs of skin aging, of a cosmetic composition comprising, as one of its active agents, at least one agent that activates or stimulates survivin expression, for the purpose of obtaining an antiwrinkle effect, through a phenomenon of cell re-densifying of the epidermis, particularly in the hollow of the wrinkles, and through the acceleration or the maintenance of the renewal thereof.

According to another embodiment of the invention, said cosmetic care method comprises the application, to areas of skin exposed to sunlight of at least a part of the skin of the face or of the body, of a composition comprising, as one of its active agents, at least one agent that activates or stimulates survivin expression, for reinforcing the resistance of the keratinocytes of the basal level of the epidermis so as to reduce the cell loss at the basal level which results from this exposure to sunlight, and thus limiting photo aging.

A second subject of the invention relates to a cosmetic care method for the care of, or for reconstructing the epidermis or the stratum corneum thereof, notably which is damaged, in particular by ultraviolet radiation, said method being characterized in that it comprises the delivery, to at least a part of the skin of the face or of the body, of an effective amount of at least one cosmetically acceptable agent that activates or stimulates survivin expression in the skin.

Said method comprises the application, to at least a part of the damaged area of the skin of the face or of the body, of a cosmetic or dermatological composition comprising, as one of its active agents, at least one agent that activates or stimulates survivin expression, for the purpose of accelerating or promoting healing of the skin.

A third subject of the invention relates to a cosmetic care method aimed at restoring the functioning of the hair cycle, in order to slow down or prevent hair loss, promote or accelerate hair regrowth, in particular in the case of alopecia, or reinforce brittle hair, said method being characterized in that it comprises the delivery, to at least a part of the scalp, of an effective amount of at least one cosmetically acceptable agent that activates or stimulates survivin expression in the skin.

According to one variant of the invention, said care method comprises the application, to at least a part of the skin of the scalp, of a cosmetic composition comprising, as one of its active agents, at least one agent that activates or stimulates survivin expression, for the purpose of obtaining the desired effect.

A fourth subject of the invention relates to a cosmetic care method for promoting growth of the nail and/or reinforcing the strength thereof, said method being characterized in that it comprises the delivery, to the nail or at least a part of the surrounding area, of an effective amount of at least one cosmetically acceptable agent that activates or stimulates survivin expression.

According to one variant of embodiment of the invention, said method comprises the application, to the nail or the surrounding area, of a cosmetic composition comprising, as one of its active agents, at least one agent that activates or stimulates survivin expression, for the purpose of obtaining the desired effect.

A fifth subject of the invention relates to a method for the in vitro culture of stem cells and/or of cells with a high clonogenic potential, for the purposes of fundamental studies or the production of cultured epidermis, such as reconstructed epidermis, for therapeutic purposes, for instance in the case of grafts following burns or ulcerative wounds that heal poorly, characterized in that it comprises the addition, to the culture medium, of an effective amount of an agent that activates or stimulates survivin expression for maintaining said cells in culture.

The cosmetically or dermatologically acceptable active agent according to the invention that activates or stimulates survivin expression may be a purified molecule, of natural or synthetic origin, or else may be the product of a method of extraction from a starting material of plant, mineral or animal origin.

This active agent may in particular be an extract or an essential oil.

According to one particular embodiment of the invention, the active agent that stimulates survivin expression is forskolin or an extract containing it, in particular an extract of Coleus forskolii.

The present invention is thus also directed towards the use of forskolin or of an extract containing it, as a cosmetic agent, for preventing or delaying the appearance of the signs of skin aging or treating them.

The other plant species of which the extraction product makes it possible to obtain the desired effect of stimulation of survivin expression are more particularly chosen from the group comprising Nostoc commune, Scenedesmus dimorphus, Curcuma longa, Crocus sativus, Daniellia oliveri, Lepechinia caulescens and Limnophila conferta.

The active agent according to the invention may be a plant extract obtained from a plant material formed from a single plant species or from a mixture of plant species belonging to the same genus or different genera, and in the freshly harvested or dried state.

Said plant extract may be obtained by any extraction method known to those skilled in the art, in particular by implementing the extraction methods described below and also in examples 2 and 3.

The plant material used for preparing the extract may be the whole plant or a part of the plant, such as the root, the rhizome or an above-ground part, in particular the stem, the leaves, the flowers, the seeds or the floral buds.

Prior to the extraction step itself, the plant material may have been dried and/or ground. According to one preferred embodiment of the extraction, the plant material is in the dry and ground state.

The extract may be prepared by various extraction methods known to those skilled in the art.

However, the extraction is in particular carried out by bringing the selected plant material into contact with a polar solvent or a mixture of polar solvents.

According to the present invention, the expression “polar solvent” signifies that the solvent has a polarity index value P′ which is greater than or equal to a value of 4. The polarity index is a value calculated on the basis of thermodynamic values (of solubility and of change of state) which reveals the more or less polar nature of a molecule. For the polarity indices of the solvents, reference will be made to the article by L. R. SNYDER; Classification of the solvent properties of common liquids; Journal of Chromatography, 92 (1974), 223-230, which is included in the present application by way of reference.

As polar solvent or mixture of polar solvents that can be used for the extraction step, a solvent chosen from water, a C₁-C₄ alcohol, preferably chosen from ethanol or butanol, a glycol preferably chosen from glycerol, butylene glycol and propylene glycol, and mixtures thereof in any proportions, will advantageously be chosen.

According to one preferred embodiment of the invention, the extracts obtained from the plant species Coleus forskolii, Nostoc commune, Scenedesmus dimorphus, Curcuma longa, Crocus sativus, Daniellia oliveri, Lepechinia caulescens and Limnophila conferta are extracts based on a polar solvent or on a mixture of polar solvents, advantageously chosen from water, a C₁-C₄ alcohol, in particular ethanol or butanol, a glycol preferably chosen from glycerol, butylene glycol and propylene glycol, and mixtures thereof.

The preferred mixtures are mixtures of at least one alcohol and of water, or of at least one glycol and of water, comprising at least 10% v/v of alcohol or of glycol, the remainder being made up of water.

According to one preferred embodiment of the method of extraction from these plant species, the extraction step per se is carried out by hot reflux, for at least 30 minutes.

The extraction may also optionally comprise an additional step comprising a treatment of the plant material or of the plant extract, aimed at partially or completely discolouring it or at purifying it. This discoloration step may, for example, comprise a treatment of the plant material or of the extract with a solution of an apolar solvent or of a mixture of apolar solvents, or a treatment consisting in bringing the extract into contact with particles of active carbon, or else a treatment using CO₂ in the supercritical state.

The extraction may be completed with a step of partial or total elimination of the extraction solvents. In the first case, the extract is generally concentrated until an aqueous concentrate devoid of significant amounts of organic solvent is obtained; in the second case, a dry residue is obtained. Alternatively, the product of the extraction step may be lyophilized or atomized so as to be in the form of a powder.

The powder may be used as it is in a cosmetic or dermatological composition according to the invention or may be redispersed in a solvent or a mixture of solvents.

In general, the product of the extraction step may be dissolved or dispersed in a solvent or a mixture of solvents, so as to be used as an active agent in the cosmetic or dermatological compositions of the invention. The solvent or the mixture of solvents in which the extract is dissolved or dispersed may be identical to or different from that having been used for the extraction.

The extract of the invention may also be adsorbed onto a support advantageously chosen from porous or nonporous nylon powders, and micas, or any lamellar mineral substance. In this case, the extract used is preferably an aqueous extract.

According to one variant of embodiment of the present invention, the agent that activates or stimulates survivin expression is delivered topically in the form of a cosmetic composition containing said agent that stimulates survivin expression as one of its active agents, said composition also comprising at least one cosmetically acceptable excipient, by application of this composition to the skin of the body or of the face, or the superficial body growths.

The cosmetic or dermatological composition according to the invention comprises an effective amount of extract of the invention for obtaining the desired effect.

The term “effective amount”, for any aspect of the invention, is intended to mean an amount which is at least equal to the amount necessary:

a) for preventing or delaying the appearance of the signs of skin aging or slowing down the effects thereof, and/or

b) for reconstructing the epidermis or the stratum corneum thereof when it is damaged, in particular by ultraviolet radiation, and/or

c) for restoring the functioning of the hair cycle, for slowing down or preventing hair loss, accelerating or promoting hair regrowth, in particular in the case of alopecia, or for reinforcing brittle hair, and/or

d) for promoting growth of the nail and/or reinforcing the resistance thereof;

e) maintaining the stem cells, and/or the cells with a high clonogenic potential, in culture, in order to make it possible to conserve these cultures for a sufficient period of time and to carry them out under good conditions, and also for carrying out the production of epidermis, when necessary.

In practice, this amount can be readily determined by those skilled in the art. By way of example, it may be indicated that the concentration of agent that activates and/or stimulates survivin production, in the composition or the culture medium, will be between 0.001% and 5% by weight.

The culture medium preferably comprises between 0.01% and 3%, by weight of the culture medium, of agent that activates and/or stimulates survivin production.

The cosmetic or dermatological composition according to the invention advantageously comprises from 1% to 3% of agent that activates and/or stimulates survivin production.

The cosmetic or dermatological composition according to the invention may also comprise one or more other cosmetically or dermatologically acceptable agents.

As demonstrated by specific tests which have been carried out and reported in examples 1 to 3, the cosmetic or dermatological agent according to the invention is effective in particular by stimulating, unexpectedly, survivin expression in the stem cells of the basal layer of the epidermis.

The tests carried out by the inventors have also shown that the properties of a cosmetic or dermatological active agent that stimulates survivin expression according to the invention can also be obtained or improved in cosmetic or dermatological compositions, in which said agent is combined with other active agents having cosmetic effects similar and/or complementary to the extract of the invention.

The activating effectiveness of a cosmetic or dermatological active agent that stimulates survivin expression according to the invention will in particular be advantageously improved by molecules or extracts that stimulate the expression of the adhesion proteins, such as beta-1 integrins, of the epidermal keratinocytes, and the adhesion itself of these cells (magnesium aspartate, manganese salts and derivatives), certain peptides recognized by the integrin, such as the arginine-glycine-aspartic acid sequence, or certain growth factors such as KGF.

The activating effectiveness of a cosmetic or dermatological active agent that stimulates survivin expression according to the invention may also be advantageously improved by molecules or extracts that inhibit phosphodiesterases which degrade cAMP, such as methylxanthines and in particular caffeine, and result in an increase in the intracellular cAMP level.

The cosmetic or dermatological compositions according to the invention may also comprise one or more other active agents that may be chosen from substances having a skin-lightening activity; substances having a slimming activity; substances having a hydrating activity; substances having a calming, soothing or relaxing activity; substances having an activity that stimulates cutaneous microcirculation so as to improve the radiance of the complexion, in particular of the face; substances having a sebum-regulating activity for greasy skin care; substances for cleansing or purifying the skin; substances having a free-radical-scavenging activity; substances for reducing or delaying the effects of skin aging, in particular the formation of wrinkles, through an activity aimed at promoting maintenance of the skin structure and/or at limiting degradation of the extracellular matrix of the superficial layers of the dermis and of the epidermis and/or at obtaining a skin-protecting, -correcting or -restructuring effect; substances having an anti-inflammatory activity.

In addition to the extract of the invention, said cosmetic or dermatological composition comprises at least one cosmetically or dermatologically acceptable excipient which may be chosen from pigments, dyes, polymers, surfactants, rheology agents, fragrances, electrolytes, pH modifiers, antioxidants and preservatives, and mixtures thereof.

The cosmetic or dermatological composition according to the invention may, for example, be a serum, a lotion, an emulsion, for example a cream, or alternatively a hydrogel, preferably a mask, or may be in the form of a stick, or else of a patch, or else a hygiene product for the scalp, such as a shampoo or a conditioner, or alternatively a make-up product, in particular a composition intended to be applied to the nails, for example a nail varnish.

Finally, the present invention concerns the use of the active agents as defined above as a cosmetic or dermatological agent for preventing or delaying the appearance of the signs of skin aging or treating them, said cosmetic or dermatological agent stimulating survivin expression in the stem cells of the basal layer of the epidermis.

The invention also relates to the use of the active agent of the invention as a cosmetic or dermatological agent or for the production of a cosmetic composition for preventing or delaying the appearance of the signs of skin aging or treating them.

Other objectives, characteristics and advantages of the invention will emerge clearly from the following explanatory description given with reference to several exemplary embodiments of the invention given simply by way of illustration and which cannot in any way limit the scope of the invention. In the examples, the temperature is in degrees Celsius, the pressure is atmospheric pressure, and the amounts or the percentages are given by weight, unless otherwise indicated.

### Examples

### Materials and Methods

1) Cell Culture

The normal human keratinocytes are cultured in 75 cm² flasks, in an incubator at 37° C. under a humid atmosphere containing 5% CO₂, in serum-free keratinocyte medium supplemented with EGF (Epidermal Growth Factor) and with BPE (Bovine Pituitary Extract) (KSFMc) (Gibco ref: 17005-034+37000-015). The cells are seeded (day D0) into 48-well microplates in a proportion of 50 000 cells in 500 μL of medium per well.

After incubation for 24 hours (day D1), the cells have become adherent and the treatment step is then carried out. The seeding medium is removed and the treatment medium containing the ingredients to be evaluated at the various concentrations, or the excipient thereof (for example DMSO) in the same proportion, is then added to each culture well.

A peak of survivin expression by the cells is observed after treatment for 16 hours. The wells are then rinsed with PBS. Half the wells of the microplate are used to lyse the keratinocytes and to assay the intracellular survivin. The other half of the wells of the microplate are used to assay the total proteins by the BCA method, which makes it possible to relate the amount of survivin assayed back to a unit amount of protein.

A phase of measuring the cytotoxicity of each active agent tested is necessary beforehand, in order to be able to subsequently evaluate the effect of the active agent at noncytotoxic doses.

To this end, the cytotoxic dose of the active agent is determined by means of the XTT test (ref: Cell Proliferation Kit II, Roche Diagnostic). The tetrazolium salt (XTT reagent) is converted to formazan by the dehydrogenases located in the mitochondrial respiratory chain. Only the living cells, the respiratory chain of which is functional, are capable of producing formazan, an orange compound detected at 450 nm.

Each active agent tested is diluted so as to prepare a doubling dilution range, the concentration of active agent of the test samples ranging from 50 mg/ml to 0.195 mg/ml. Each pre-prepared dilution is finally diluted to 1/1000th in KSFM-C medium and then brought into contact with the keratinocytes for 48 h, the time at which the cytotoxicity test will be carried out.

### 2) Assaying of Survivin

The survivin is assayed using an ELISA enzymatic immunoassay (ref: Duoset Survivin ELISA from R&D Systems) on cultures of normal human keratinocytes.

The total proteins are assayed using a BCA colorimetric test (reference: BC Assay Kit, Uptima Interchim), by measuring absorbance at 570 nm.

For assaying survivin by ELISA after 16 h of treatment, the wells are rinsed with PBS and then 100 μl/well of lysis buffer are added, followed by incubation for 10 minutes with gentle shaking. This buffer contains antiproteases, which prevent degradation of the proteins, including survivin, during the cell lysis.

The ELISA microplate is prepared (reference Clear Microplate R&D systems DY992):

A standard range with human survivin is prepared from 0 to 2000 pg/ml under the same conditions as with the cell lysates.

After the enzyme reaction has been blocked with sulfuric acid, the survivin is quantified by measuring absorbance at 450 nm.

### Example 1

### Activity of Forskolin on Survivin Expression

The forskolin, which is commercially available from the supplier SIGMA, France, is indicated as having been isolated from an extract of the plant Coleus forskolii.

The forskolin is first of all diluted in DMSO so as to prepare a solution at 4 mg/ml.

During the treatment on cells, the previously prepared solution is diluted in the culture medium so as to obtain a final concentration of 10 μM, i.e. 4 μg/ml. A control is also prepared using this same solvent and in the same proportions.

The result, indicated in table I below, is compared with the basal level of survivin expression, represented by a solvent control which constitutes 100%:

TABLE I

| | Dose | Survivin | |
|---|---|---|---|
| Active agent | (μg/ml) | (pg/mg proteins) | % activation |
| Solvent control | — | 4.92 | 100 |
| Forskolin | 4 | 7.27 | 147.9 |

Conclusion: forskolin significantly increases intrakeratinocyte survivin expression, compared with the basal level of expression of the protein (controls).

### Example 2

### Preparation of an Extract of Ground Parts of Limnophila conferta and Determination of the Activity Thereof with Respect to Survivin Expression

A—Preparation of the Extract

The plant material, which is commercially available from the supplier IDVP, France, formed from ground parts comprising the stems and leaves of Limnophila conferta in the dry state, is ground extemporaneously using a laboratory mill-mixer, to an average particle size of the order of 0.1 to 1 mm.

10 g of ground plant material are introduced into a 250 ml round-bottomed flask, to which 150 ml of an ethanol/water mixture (90/10 v/v) are added.

The round-bottomed flask surmounted by a bulb condenser is magnetically stirred in a thermostatic bath, and then heated to the reflux of the solvent.

The reflux is maintained for 30 min with stirring.

Once the heating has stopped, the round-bottomed flask is left to cool to ambient temperature outside the bath.

The mixture is then vacuum-filtered through a büchner funnel with a 70 μm Whatman GF/F filter and a tared flask; the filtrate 1 is thus obtained. The cake is washed on the büchner funnel with 50 ml of the extraction solvent; the filtrate 2 is obtained.

The two filtrates are then combined and weighed.

The resulting filtrate is introduced into a pre-tared round-bottomed flask, and then concentrated to dryness in a rotary evaporator under vacuum in a bath of water at a maximum temperature of 50° C.

The dry residue is quantified in order to determine the extraction yield by mass, expressed as mass of dry extract obtained per 100 g of starting plant material in the dry ground state.

The extraction yield is 21%.

B—Activity with Respect to Survivin Expression

The dry extract prepared in paragraph A is diluted to the concentration of 12.5 mg/ml or 25 mg/ml in DMSO.

During the treatment on cells, the extract is added to the culture medium in order to obtain a final concentration of 0.1% v/v, i.e. 12.5 μg/ml for the first solution of extract and 25 μg/ml for the second solution. A control is also prepared using this same solvent, with a final concentration of 0.1% v/v.

Table II below indicates the average activity of the extract of example 1 with respect to survivin expression, expressed as picograms of survivin per mg of total proteins, and then compared with the basal level of expression of this same protein, represented by the solvent control, which constitutes 100%.

The results obtained are indicated in table II below:

TABLE II

| | Dose | Survivin | |
|---|---|---|---|
| Plant | (μg/ml) | (pg/mg proteins) | % activation |
| Solvent control | — | 600 | 100 |
| Limnophila conferta | 12.5 | 776 | 129.3 |
| Limnophila conferta | 25 | 889 | 148.1 |

Conclusion: the extract of Limnophila conferta significantly increases intrakeratinocyte survivin expression, compared with the basal level of expression of the protein (controls).

### Example 3

### Preparation of an Extract of Ground Parts of Lepechinia caulescens and Determination of the Activity Thereof with Respect to Survivin Expression

A—Preparation of the Extract

The extract is prepared in accordance with example 2-A, using Lepechinia caulescens leaves in the dry and ground state as starting plant material.

The extraction solvent used in the method is a 50/50 (v/v) ethanol/water mixture.

The dry residue is quantified in order to determine the extraction yield by mass, expressed as mass of dry extract obtained per 100 g of starting plant material in the dry ground state.

The extraction yield by mass obtained is 29%.

B—Activity with Respect to Survivin Expression

The dry extract prepared in paragraph A of the present example is diluted to the concentration of 3.125 mg/ml in DMSO.

During the treatment on cells, the extract is added to the culture medium in order to obtain a final concentration of 0.1% v/v, i.e. 3.125 μg/ml. A solvent control (DMSO) with a final concentration of 0.1% v/v is also prepared.

Table II below indicates the activity of the extract of Lepechinia caulescens tested with respect to survivin, compared with the basal level of expression, represented by the solvent control which constitutes 100%:

TABLE III

| | Dose | Survivin | |
|---|---|---|---|
| Plant | (μg/ml) | (pg/mg proteins) | % activation |
| Solvent control | — | 4.92 | 100 |
| Lepechinia caulescens | 3.125 | 6.57 | 133.7 |

Conclusion: the extract of Lepechinia caulescens significantly increases intrakeratinocyte survivin expression, compared with the basal level of expression of the protein (controls).

### Example 4

### Cosmetic Composition Comprising an Extract of Lepechinia caulescens Leaves

The dry extract obtained in example 3A is solubilized at 1% by mass in an ethanol/water mixture.

A solution at 1% by mass of dry extract is obtained and is used in the cosmetic composition below:

| | |
|---|---|
| Plant extract of Lepechinia caulescens (EX3A) | 0.1% |
| Surfactant (Arlacel ® 165 VP) | 5% |
| 95% cetyl alcohol | 1% |
| Stearyl alcohol | 1% |
| Beeswax | 1.5% |
| Oil (Perleam ®) | 8.5% |
| Tri caprate/caprylate glycerides | 3% |
| Silicone oil (dimethicone 100 CS) | 1% |
| Polymer (Keltrol ®) | 0.35% |
| Sodium hydroxide | 0.04% |
| Tetrasodium EDTA powder | 0.1% |
| Preservatives | 0.5% |
| Water | qs 100 |

The cosmetic composition is prepared in the usual manner, well known to those skilled in the art, by mixing the various components in one or more steps.

This composition can be applied daily to the skin or the scalp or else the nails, for several weeks, so as to obtain the cosmetic effects indicated above.

### Example 5

### Cosmetic Composition Comprising Forskolin

The forskolin of example 1 (origin SIGMA) isolated from an extract of Coleus forskolii, is used in the cosmetic composition below:

| | |
|---|---|
| Forskolin (SIGMA, EX 1) | 2% |
| Steareth-21 (Brij 721) | 2.5% |
| Glyceryl stearate (Tegrin) | 1.1% |
| Stearyl alcohol | 5% |
| Glycerol tricaprate/caprylate | 12.5% |
| Butylene glycol | 3% |
| Glycerol | 2% |
| Preservative | 0.5% |
| Fragrance concentrate | 0.5% |
| UV screen (octyl methoxycinnamate) | 7.5% |
| Water | qs 100 |

The cosmetic composition is prepared in the usual manner, well known to those skilled in the art, by mixing the various components in one or more steps.

This composition can be applied daily to the areas of the skin comprising wrinkles, for several weeks, so as to obtain an effect of reduction or of complete disappearance of said wrinkles.

What is claimed:

1. A method for caring for a body zone in need thereof, comprising:
delivering, to at least a part of the body zone, an effective amount of at least one cosmetically acceptable agent that activates or stimulates survivin expression in said body zone.

2. A method for preventing or delaying the appearance of the signs of skin aging; reducing the effects of skin aging; caring for the epidermis; or caring for stratum corneum, comprising:
delivering, to at least a part of the skin of the face or of the body, an effective amount of a cosmetically acceptable agent that activates or stimulates survivin expression in the skin.

3. A method for restoring the functioning of the hair cycle, for accelerating or promoting hair regrowth, or reinforcing brittle hair comprising:
delivering, to at least a part of the scalp, an effective amount of at least one cosmetically acceptable agent that activates or stimulates survivin expression in the scalp.

4. A method for promoting growth of a nail, for reinforcing the strength of a nail, or both, comprising:
delivering, to the nail or at least a part of the area surrounding the nail, an effective amount of at least one cosmetically acceptable agent that activates or stimulates survivin expression.

5. The method according to claim 1, wherein said agent is delivered topically in the form of a cosmetic composition comprising said agent as an active agent, said composition further comprising at least one cosmetically acceptable excipient.

6. The method according to claim 1, wherein the cosmetically active agent is chosen from the group consisting of forskolin or an extract containing forskolin, an extract of Lepechinia caulescens, an extract of Limnophila conferta, an extract of Daniellia oliveri, an extract of Nostoc commune, an extract of Scenedesmus dimorphus, an extract of Curcuma longa, and an extract of Crocus sativus.

7. The method according to claim 1, wherein the cosmetically active agent comprises an extract of Coleus forskolii.

8. The method according to claim 1, wherein the cosmetically active agent comprises an extract of Lepechinia caulescens.

9. The method according to claim 1, wherein the cosmetically active consists essentially of an extract of Lepechinia caulescens.

10. The method according to claim 1, wherein the cosmetically active agent comprises an extract of Limnophila conferta.

11. The method according to claim 1, wherein the cosmetically active agent consists essentially of an extract of Limnophila conferta.

12. The method according to claim 5, wherein the concentration of cosmetically active agent is between 0.001% and 5%, by weight of the composition.

13. The method according to claim 5, wherein the concentration of the cosmetically active agent is from 1% to 3%, by weight of the composition.

14. The method according to claim 1, wherein the agent that activates or stimulates survivin expression is combined with a molecule or extract that stimulates the expression of adhesion proteins, of epidermal keratinocytes, or the adhesion itself of these cells.

15. The method according to claim 14, wherein the adhesion protein is a beta-1 integrin.

16. The method according to claim 14, wherein the molecule or extract is magnesium aspartate, a manganese salt or derivative, peptides recognized by beta-1 integrin, or growth factors.

17. The method according to claim 16, wherein the peptide recognized by beta-1 integrin has the arginine-glycine-aspartic acid sequence.

18. The method according to claim 16, wherein the growth factor is KGF.

19. The method according to claim 1, wherein the agent that activates or stimulates survivin expression is combined with a molecule or with an extract that inhibits diphosphoesterases.

20. The method according to claim 19, wherein the molecule is caffeine.

21. The method according to claim 5, wherein the cosmetic composition is formulated in a form selected from the group consisting of a serum, a lotion, an emulsion, a hydrogel, a stick, a patch, a hygiene product for the scalp, a shampoo, a conditioner, a make-up product, a composition intended to be applied to the nails, and a nail varnish.
